(19) United States
(12) Patent Application Publication
(10) Pub. No.: US 2021/0069095 A1
(43) Pub. Date: Mar. 11, 2021

(54) LEAVE-ON HAIR CARE COMPOSITIONS

(71) Applicant: L'OREAL, Paris (FR)

(72) Inventors: João Paulo Duarte GUIMARAES, Rio de Janeiro (BR); Leandro Valeriano DE CARV ALHO, Rio de Janeiro (BR); Alice Matos XAVIER, Rio de Janeiro (BR); Danielle Pestana PANTOJA, Rio de Janeiro (BR); Laura De Carvalho E SILVA, Rio de Janeiro (BR); Ana Claudia Meda SILOTO, Rio de Janeiro (BR); Diego Fernando Valio Antunes ALVES, Säo Paulo (BR)

(21) Appl. No.: 16/772,366

(22) PCT Filed: Dec. 15, 2017
(86) PCT No.: PCT/BR2017/050384

§ 371 Date: Jun. 12, 2020

(51) Int. Cl. A61K 8/92; A61K 8/34; A61K 8/41; A61K 8/73; A61K 8/06; A61K 8/891; A61K 8/892; A61K 8/58; A61Q 5/12

(52) U.S. Cl. CPC A61K 8/922; A61K 8/342; A61K 8/416; A61K 8/737; A61Q 5/12; A61K 8/891; A61K 8/892; A61K 8/585; A61K 8/062

(57) ABSTRACT

The present invention is directed to a leave-on hair care composition and a hair treatment agent employing the leave-on hair care composition, mainly for curly hair. The leave-on hair composition comprises high concentrations of cationic polymer associated with a fatty alcohol, vegetal oil and at least two cationic surfactants, its uses and method for controlling the frizz.

### FIELD OF THE INVENTION

The present invention is directed to a leave-on hair care composition and a hair treatment agent employing the leave-on hair care composition, mainly for curly hair. The leave-on hair composition comprises high concentrations of cationic polymer associated with a fatty alcohol, vegetal oil and at least two cationic surfactants, its uses and method for controlling the frizz.

### BACKGROUND OF THE INVENTION

People have been using a number of cosmetic and personal care compositions to enhance the look and feel of keratin fibers, such as the hair. Exposure to humid conditions and chemical procedures can cause an excessively expanded volume (i.e., “frizz volume”), or appear frizzy. This frizzy condition of hair often appears unruly and is often characterized with a multiplicity of fly-away strands of hair. Frizzy hair is unsightly and makes it difficult for consumers to control and maintain the hair style. This frizzy condition is particularly pronounced in dry, damaged or curly hair.

Curly hair tends to become frizzy and significantly expand in volume when exposed to high humidity conditions or changes in humidity environment.

For temporary control, styling mousse, gels, lotions, and spray compositions have been employed to combat these conditions.

Many attempts of compositions have been developed in order to conditioning, discipline and control the hair, in particular curly hair, enhancing/maintaining curl definition, curl retention, and curl regularity, and/or enhancing shine, without leaving the hair feeling and/or looking unnatural and stiff. Unfortunately, the products of the state of the art usually leave the hair stiff and unmanageable after application and negatively affect ease of combing and hair feel.

Furthermore, according to U.S. Pat. No. 9,622,945, many compositions have been proposed to overcome the frizz effect of the hair. A conventional approach to reducing the frizzy appearance of hair is by applying silicones to the hair. However, the use of silicones alone is often impractical, because while they may effectively reduce the frizzy appearance of hair, they can also weigh down the hair and/or cause the hair to feel greasy and tacky. Conventional compositions may also flake off or leave unsightly residues or unwanted product buildup.

Therefore, it is desirable for manufacturers to formulate a leave-on hair care composition with light texture to treat hair as well as provide such a composition in a hair treatment agent or system without the drawbacks described above, and that can impart superior frizz control, curl definition, and hair discipline, without weighing down the hair.

### SUMMARY OF THE INVENTION

The inventors of the present invention achieved this objective through a leave-on hair care composition with good substantivity for the hair and that reduces hair frizz and controls hair volume, as well as hair treatment agents and methods employing said leave-on hair care composition for controlling the frizz.

Such a leave-on composition has a light texture, and mainly for curly hair, comprises a cationic polymer in high concentrations associated with a fatty alcohol, vegetal oil and at least two cationic surfactants.

In other words, such specific combination of high concentration of cationic polymer and the vegetal oil will result in a composition with light feel that forms a film on the hair and is able to control the frizz and improve the visual aspect of the curls (more curl definition, curl regularity, and discipline).

### BRIEF DESCRIPTION OF THE DRAWINGS

The accompanying drawings, which are included to provide a further understanding of the invention and are incorporated in and constitute a part of this specification, together with the description, serve to explain the principles of the invention.

FIG. 1 is a photograph of a comparative test between a composition of the state of the art without the cationic polymer, a composition of the present invention with the cationic polymer and a non-treated control showing the final aspect of the hair (swatches) after applying such compositions in conditions of (i) just after drying in room temperature, (ii) just after drying in room conditions after passing the fingers once through the hair, and (iii) 24 h after passing the fingers through the hair (swatches of caucasian hair type IV).

FIG. 2 is a photograph of the visual aspect of the hair care composition of the present invention in view of the compositions of the state of the art.

### DETAILED DESCRIPTION OF THE INVENTION

The present invention is directed to a leave-on hair care composition having light texture, mainly for curly hair, comprising a cationic polymer in high concentrations associated with a fatty alcohol, vegetal oil and at least two cationic surfactants, its uses and methods for controlling the frizz.

The leave-on hair care composition of the present invention comprises:

(a) about 0.9 to about 1.5% by weight of a cationic polymer;

(b) about 0.8 to about 5% by weight of a vegetal oil;

(c) about 3 to about 7% by weight of a fatty alcohol; and

(d) about 0.35 to about 5% by weight of at least two cationic surfactants;

all weights relative to the total weight of the composition.

The leave-on hair care composition according to the invention provides light texture to treat the hair, mainly for curly hair.

Also, the leave-on hair care composition of the present invention is directed to control the frizz of the hair and curl definition.

The pH of the leave-on hair care composition of the invention is preferably of about 4.5 and may range from about 3.5 to about 4.5.

The density of the leave-on hair care composition of the invention is preferably within the range of about 0.9 g/cm³ to about 1.0 g/cm³, and is more preferably, of about 1 g/cm³.

The viscosity of the leave-on hair care composition of the invention is preferably of about 1.685 mPa·s and may range from about 1.055 mPa·s to about 1.895 mPa·s.

In a preferred embodiment, the leave-on hair care composition of the invention uses a ratio of cationic polymer/vegetal oil ranging from about 1:1 to about 1:2, including all ranges and subranges therebetween, or is at a ratio of about 1:1 or about 1:1.5.

In an embodiment, when the two cationic surfactants are behentrimonium chloride and ceterimonium chloride, the weight ratio of behentrimonium chloride to cetrimonium chloride is from about 1.5:1 to about 0.1:1, such as from about 1.25:1 to about 0.3:1, or such as from about 1:25:1 to about 0.4:1. In some embodiments, the weight ratio of behentrimonium chloride to cetrimonium chloride is from about 1.25:1 to about 1:1. In some embodiments, the weight ratio of behentrimonium chloride to cetrimonium chloride is from about 0.9:1 to about 0.4:1.

The leave-on hair care composition of the invention is preferably in the form of an oil in water (O/W) emulsion.

In another preferred embodiment, the composition of the invention is for the manufacturing of a product to be used as a leave-on on the hair to control the frizz, improve the curl definition, curl regularity, discipline, among others attributes.

Moreover, the present invention is directed to a method for controlling the frizz of curly hair, wherein the method comprises contacting the hair with the composition.

In addition, the leave-on hair care composition of the invention can be used as a daily product for the hair, preferably in traditional routines, since as will be demonstrated, different routines customized with other types of products would not enhance the benefits described above.

A traditional routine includes the use of the leave-on hair care composition of the present invention after a shampoo and conditioner.

Different types of routines are related to the use of the leave-on hair care composition of the invention after the shampoo and conditioner, jointly or after a serum or when it is included in a mask before such routine.

In another embodiment of the present disclosure, the performance of the hair care composition of the present invention (Example 1) was compared with three examples (A to C): (A) without guar and with coconut oil, (B) with guar and mineral oil but without coconut oil and (C) a benchmark composition (Example 7).

### Terms

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

### Leave-On Hair Care Composition

### Cationic Polymer

Non limiting examples of cationic polymer that can be used in the leave-on hair care compositions of the present invention include quaternary ammonium guar such as including particularly guar hydroxypropyltrimonium chloride; polymer derivatives of diallyldimethyl ammonium chloride (“poly-DADMAs”) and of methacrylamido-propyltrimethylammonium chloride (“poly-MAPTACs”), and mixtures thereof.

Non-limiting examples of poly-DADMAs and poly-MAPTACs include, polyquaternium-4, polyquaternium-5, polyquaternium-6, polyquaternium-7, polyquaternium-22, polyquaternium-37, polyquaternium-39, polyquaternium-47, polyquaternium-53, and mixtures thereof.

In preferred embodiments, the at least one cationic polymer of the present invention is chosen from guar hydroxypropyltrimonium chloride which is commercially available under the trade name N-HANCE from the company Ashland or JAGUAR C-13-S from the company Rhodia (Solvay).

In various embodiments, the concentration of the cationic polymer may range from about 0.9% to 1.5% by weight, such as from about 1.0% to about 1.5% by weight, or preferably from about 1.0% to about 1.2%, including all ranges and subranges therebetween, relative to the total weight of the composition.

Such amounts are considered high concentrations of cationic polymer when compare to the ones from the state of the art, which usually uses up to about 0.98% by weight of the total weight of the composition.

### Vegetal Oil

Non-limiting examples of plant-based or vegetal oils include non-limiting example include plant-based or vegetal oils such as acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil.

In some embodiments, the suitable vegetal oils for use in the leave-on hair care compositions of the present invention are selected from coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, almond oil, babassu oil (Orbigyna Oleifera Seed Oil), jojoba oil, peanut oil, soybean oil, helianthus annuus (sunflower) seed oil, and mixtures thereof.

In some cases, the leave-on hair care composition includes at least coconut oil.

In a preferred embodiment the suitable vegetal oil of the present invention is selected from coconut oil, which may be commercially available under various tradenames from different companies such as NEUTRESCA 51-25 from the company Aarhuskarl Shamn or REFINED COCONUT OIL-L from the company Aakkamani Private or AGRIPURE AP-20 from the company Cargill or EDIBLE COCONUT OIL N° 76 from the company Welch Holme & Clark.

The vegetal oil is used with the objective to confer softening and moisturizing to the hair since it forms a hydrophobic film on the hair fiber. Also, the leave-on hair care composition of the present invention improves the curl definition, curly regularity, discipline, among others attributes, without weighting down the hair.

The vegetal oil may be present at a concentration from about 0.8% to about 5%, preferably from about 1% to about 3% and more preferably from between about 1.0% to about 2% by weight, including all ranges and subranges therebetween, relative to the total weight of the composition.

### Fatty Alcohol

The fatty alcohol corresponds to linear, branched saturated/unsaturated fatty alcohols comprising from 6 to 60 carbon atoms and preferably correspond to the formula R-OH in which R is a saturated or unsaturated, linear or branched hydrocarbon-based radical, comprising 6 to 60 carbon atoms, or from 10 to 50 carbon atoms, or from 12 to 24 carbon atoms, or from 10 to 22 carbon atoms optionally comprising one or more OH groups.

The saturated fatty alcohols are preferably branched and can be in liquid form. They can optionally comprise, in their structure, at least one aromatic or non-aromatic ring. They are preferably acyclic.

The unsaturated fatty alcohols exhibit, in their structure, at least one double or triple bond and preferably one or more double bonds. When several double bonds are present, there are preferably 2 or 3 of them and they can be conjugated or unconjugated. These unsaturated fatty alcohols can be linear or branched. They can optionally comprise, in their structure, at least one aromatic or non-aromatic ring. They are preferably acyclic.

The fatty alcohols may be selected from liquid fatty alcohols, solid fatty alcohols, or mixtures thereof.

Liquid fatty alcohols may be selected, for example, from octyldodecanol, 2-butyloctanol, 2-hexyldecanol, 2-undecylpentadecanol, oleyl alcohol, linoleyl alcohol, isostearyl alcohol, undecylenyl alcohol, linolenyl alcohol, behenyl alcohol, and mixtures thereof.

The fatty alcohols of the invention may be in solid form and may be non-oxyalkylenated and/or non-glycerolated. These fatty alcohols may be constituents of animal or plant waxes.

In an embodiment, the solid fatty alcohols of the present invention may be chosen from myristyl alcohol, cetyl alcohol, stearyl alcohol, cetearyl alcohol, and mixtures thereof,

Other suitable examples of the solid fatty alcohol of the present invention include branched solid fatty alcohols chosen from 2-dodecylhexadecanol, 2-tetradecyl-1-octadecanol, 2-tetradecyl-1-eicosanol, 2-hexadecyl-1-octadecanol and 2-hexadecyl-1-eicosanol, and mixtures thereof.

The solid fatty alcohol may represent a mixture of fatty alcohols, which means that several species of fatty alcohol may coexist, in the form of a mixture, in a commercial product. One example of such a commercial product is cetearyl alcohol, a mixture of cetyl alcohol and stearyl alcohol, which is commercially available under various trade names from different companies such as LANETTED or LANNETTE D/MB or LANETTE O OR LANETTE O OR/MB from the company BASF or ECOROL 68/30 or ECOROL 68/50 from the company Ecogreen Oleachemicals. Cetyl alcohol may also be commercially available under the tradename of LANETTE 16 from the company BASF.

In one embodiment, the fatty alcohol is selected from linear saturated/unsaturated fatty alcohols comprising from 6 to 60 carbon atoms and preferably from cetyl alcohol, stearyl alcohol, cetearyl alcohol (mixture of cetyl alcohol and stearyl alcohol), octyldodecanol, isostearyl alcohol, 2-hexyl decanol, palmityl alcohol, myristyl alcohol, stearyl alcohol, lauryl alcohol, oleic alcohol (or oleyl), linoleyl alcohol (or linoley-ether), linolenic alcohol (or linolenyl) and undecylenic alcohol, and mixtures thereof.

In one embodiment of the present invention, the fatty alcohol comprises cetyl alcohol and stearyl alcohol or cetearyl alcohol.

In another embodiment of the present invention, the fatty alcohol is chosen from cetyl alcohol, stearyl alcohol, cetearyl alcohol, and mixtures thereof., preferably, when the fatty alcohol in the composition of the invention is a mixture of cetyl alcohol and steary alcohol or is selected from cetearyl alcohol, ratio of cetyl alcohol to stearyl alcohol is about 30/70 or about 50/50.

The leave-on hair care composition according to the invention comprises the fatty alcohol in a range from about 4.3% to about 7% by weight, or from about 4.0% to about 6.5% by weight, or preferably from about 4.2% to about 6.0% by weight, including all ranges and subranges therebetween, relative to the total weight of the composition.

### Cationic Surfactants

The leave-on hair care composition according to the invention comprise at least two cationic surfactants.

In general, a cationic surfactant may bear one or more positive permanent charges or may contain one or more cationizable functions in the composition according to the invention. The cationic surfactants of the invention are those bearing one or more positive permanent charges, i.e. one or more quaternized nitrogen atoms.

As examples of cationic surfactants that may be used in the composition according to the invention, mention may be made of:
quaternary ammonium salts such as tetraalkylammonium chlorides, for instance dialkyldimethylammonium chlorides or alkyltrimethylammonium chlorides, in which the alkyl radical comprises from about 12 to 22 carbon atoms, in particular behenyltrimethylammonium chloride, distearyldimethylammonium chloride, cetyltrimethylammonium chloride, or benzyldimethylstearylammonium chloride, or, on the other hand, distearoylethylhydroxyethylmethylammonium methosulfate, dipalmitoylethylhydroxyethylammonium methosulfate or distearoylethylhydroxyethylammonium methosulfate, or else, finally, palmitylamidopropyltrimethylammonium stearamidopropyldimethyl(myristyl acetate)ammonium chloride sold under the name CERAPHYL 70 by the company Van Dyk.
quaternary di- or triammonium salts such as for example, FINQUAT CT-P sold by the company Finetex (Quatermum-89) and FINQUAT CT sold by the company Finetex (Quatemium-75),
quaternary ammonium salts containing at least one ester function such as diacyloxyethyldimethylammonium, diacyloxyethylhydroxyethyl methylammonium, monoacyloxyethyldihydroxyethylmethylammonium, triacyloxyethylmethylammonium monoacyloxyethylhydroxyethyldimethylammonium salts (chloride or methyl sulfate in particular), and mixtures thereof.

In a preferred embodiment, the suitable cationic surfactants according to the present invention include behentrimonium chloride and cetrimonium chloride which are preferably known as VARISOFT BT 85/Evonik and DEHYQUART A-CA/Basf, respectively.

The cationic charge such as cationic surfactants improve detangling, moisturizing and smoothness, presenting the function of a conditioning agent.

The amount of cationic surfactants employed in the present disclosure may be from about 0.35% to about 5% by weight, from about 0.4% to about 4% by weight, from about 0.45% to about 3% by weight, about 0.7 to about 2.5% by weight, including all ranges and subranges therebetween, relative to the total weight of the composition.

### Additional Ingredients

In addition to the essential components described hereinbefore, the leave-on hair care composition of the present disclosure may further comprise any usual cosmetically acceptable ingredient, which may be chosen especially from such as perfume/fragrance, polymers, preserving agents, solvents, actives, surfactants, fat materials, vitamins, vegetal extracts, emollients, non-silicone oils and non-plant/vegetal oils such as hydrocarbon oils, pH-adjusting, softeners, humectants, opacifiers, stabilizers, emollients, silicones, active agents and mixtures thereof.

A person skilled in the art will take care to select the optional additional ingredients and/or the amount thereof such that the advantageous properties of the composition according to the invention are not, or are not substantially, adversely affected by the envisaged addition.

The compositions according to the disclosure may include one more of an emollient, including polar and/or non-polar emollients. Suitable polar emollients including, but are not limited to, polyol esters and polyol ethers such as linear or branched chained polyglycerol esters and polyglycerol ethers. Particularly suitable emollients for use as the first emollient include caprylic/capric triglyceride, isopropyl alcohol, caprylic/capric glycerides, isopropyl myristate, PPG-3 myristyl ether, isopropyl palmitate, dibutyl adipate, propyleneglycol dicaprylate/dicaprate, cocoglyceride, cetearyl isononanoate, isopropyl myristate, isodecyl neopentanoate, tridecyl neopentanoate, C₁₂₋₁₅ alkyl benzoate, isopropyl lauroyl sarsosinate, phenethyl benzoate and mixtures thereof.

Non-limiting example of preserving agent which can be used in accordance with the invention include salicylic acid, chlorhexidine dihydrochloride and sodium benzoate.

Suitable solvents include, but are not limited to water, alcohols, glycols and polyols such as glycerin, caprylyl glycol, pentylene glycol, propylene glycol, butylene glycol, and mixtures thereof.

Suitable additional actives include, but are not limited to BHT, sodium hydroxide, citric acid and mixtures thereof.

Exemplary of additional vegetal extracts of the present invention include, but are not limited to, rosa canina flower extract, nelumbo nucifera flower extract, linum usitatissimum (linseed) flower extract, chamomilla recutita (matricaria) flower extract, gardenia taitensis flower and mixtures thereof.

Non-limiting examples of vitamins of the present invention include tocopherol, tocopheryl acetate, and mixtures thereof.

Non-limiting example of silicones suitable for the leave-on hair care composition of the present invention include aminosilicones such as amodimethicone.

Non-limiting suitable multivalent carboxylic acid includes is fumaric acid.

The additional ingredients may represent from about 60% to about 95%, such as from about 60%to about 92% or such as from about 65 to about 85% by weight, including all ranges and subranges therebetween, relative to the total weight of the composition.

The leave-on hair care composition can be packaged in a kit or article of manufacture that can further comprise one or more of a hair shampoo containing at least one anionic surfactant; a hair conditioner containing at least one cationic surfactant, at least one surfactant chosen from nonionic surfactants, amphoteric surfactants, and mixtures thereof; and at least one fatty alcohol; and a hair mask containing at least one conditioning agent; at least one surfactant chosen from nonionic surfactants; and at least one fatty alcohol. A hair conditioner is generally used on hair after the hair is treated with a hair shampoo and rinsed. A hair mask can be used on hair after the hair is treated with a hair conditioner and rinsed or after the hair is treated with a hair shampoo and rinsed.

The leave-on hair composition can be applied onto hair after the hair is treated with the hair shampoo and/or the hair conditioner and/or the hair mask.

The leave-on hair composition can be applied onto hair which has not been pre-treated with the hair shampoo and/or the hair conditioner and/or the hair mask.

### Serum Composition

The serum composition that can be used in a hair care treatment system or agent that employs the leave-on hair care composition of the present disclosure contains at least one silicone in an amount of from about 80 to about 100% by weight, such as from about 85 to about 99% by weight, or such as from about 88 to about 98% by weight, relative to the total weight of the serum composition.

The at least one silicone in the serum composition may be selected from dimethicone, dimethiconol, cyclopentasiloxane, amino silicones such as amodimethicone, and mixtures thereof.

The serum composition is mixable with the leave-on hair care composition of the present disclosure, such that the mixing of the two compositions results in a hair care treatment agent or hair treatment agent that can be applied onto hair, in particularly, curly hair.

Thus, in an embodiment, the serum composition is mixed with the leave-on hair care composition prior to application to hair, resulting in a hair treatment agent which is then applied onto hair, and preferably left on the hair (i.e., it is not rinsed off the hair for at least a few hours).

In an embodiment, when the serum composition is mixed with the leave-on hair care composition in order to form a hair treatment agent prior to application to hair, the weight ratio of the serum composition to the leave-on hair care composition ranges from about 3:1 to about 1:3, or such as from about 2:1 to about 1:2, including all ranges and subranges therebetween, or the weight ratio can be at about 1:1, 1:1.5, 1:2, 1:2.5, or 1:3.

In an embodiment, the serum composition is applied on hair that has been treated with the leave-on hair care composition.

In an embodiment, the serum composition is applied on hair before the leave-on hair care composition is applied onto hair.

The serum composition and the leave-on hair care composition can be packaged separately in a kit or article of manufacture for providing a hair care system, with instructions of using the compositions either individually on hair, sequentially applying them on hair, or mixing them before applying onto hair.

The kit or article of manufacture can further comprise one or more of a hair shampoo containing at least one anionic surfactant; a hair conditioner containing at least one cationic surfactant, at least one surfactant chosen from nonionic surfactants, amphoteric surfactants, and mixtures thereof; and at least one fatty alcohol; and a hair mask containing at least one conditioning agent; at least one surfactant chosen from nonionic surfactants; and at least one fatty alcohol.

### Additional Ingredients

In addition to the essential components described for the serum composition, the serum composition of the present disclosure may further comprise any usual cosmetically acceptable ingredient, which may be chosen especially from such as perfume/fragrance, polymers, preserving agents, solvents, actives, surfactants, fat materials, vitamins, vegetal extracts, non-silicone oils such as plant/vegetal oils and hydrocarbon oils, emollients, pH-adjusting, softeners, humectants, opacifiers, stabilizers, emollients, active agents and mixtures thereof.

A person skilled in the art will take care to select the optional additional ingredients and/or the amount thereof such that the advantageous properties of the serum composition according to the invention are not, or are not substantially, adversely affected by the envisaged addition.

By way of non-limiting illustration, the invention will now be described with reference to the following examples.

### EXAMPLES 1 to 3

A suitable composition of the invention is as Examples 1 to 3, as follows:

| INGREDIENT | EX. 1 | EX. 2 | EX. 3 |
|---|---|---|---|
| BEHENTRIMONIUM CHLORIDE | 0.47 | 0.47 | 0.47 |
| TRIDECETH-6 | 0.025 | — | — |
| CETRIMONIUM CHLORIDE | 0.38 | 0.38 | 0.47 |
| GUAR HYDROXYPROPYLTRIMONIUM | 0.98 | 0.98 | 0.98 |
| CHLORIDE | | | |
| CETEARYL ALCOHOL | 4.2 | 4.2 | 4.2 |
| AMODIMETHICONE | 0.3 | — | — |
| HELIANTHUS ANNUUS (SUNFLOWER) | 0.01 | — | — |
| SEED OIL | | | |
| COCOS NUCIFERA (COCONUT) OIL | 1.51 | 0.95 | 0.98 |
| ORGANIC SOLVENT (ISOPROPYL | 0.61 | 0.61 | 0.61 |
| ALCOHOL, GLYCERIN) | | | |
| PLANT/VEGETABLE EXTRACTS, | 0.12 | — | — |
| VITAMINS (TOCOPHEROL) AND | | | |
| OTHER ADDITIVES (ESTERS, | | | |
| GLYCERIDES) | | | |
| PRESERVATIVES, | 0.91 | 0.69 | 0.09 |
| FRAGRANCE, pH | | | |
| ADJUSTERS | | | |
| WATER | QS | QS | QS |

### EXAMPLE 4

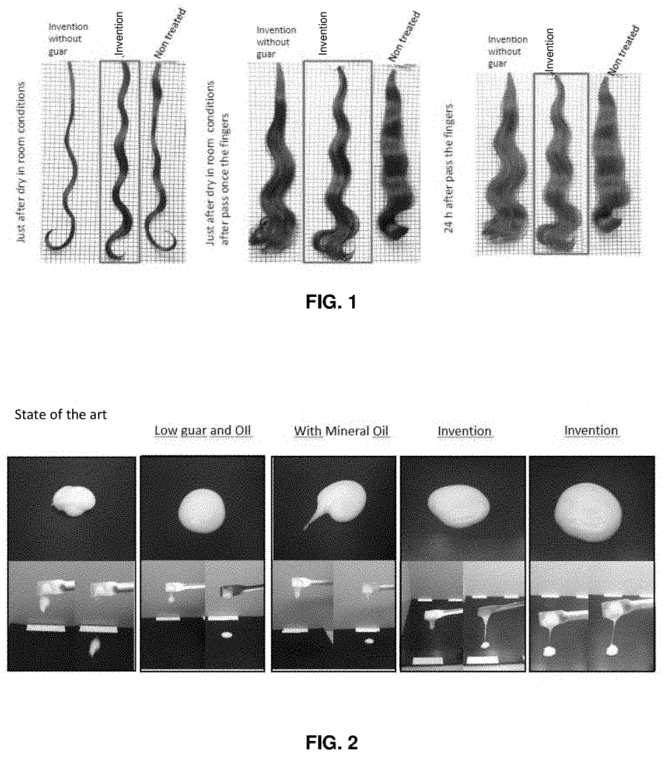

A test was performed in order to compare the performance of a composition of the state of the art without cationic polymer and comprising a mineral oil, the composition of the present invention, and a non-treated control on swatches of caucasian hair type IV. The swatches were compared after applying such compositions in the conditions of (i) just after drying in room temperature, (ii) just after drying in room conditions after passing the fingers once through the hair, and (iii) 24 h after passing the fingers through the hair (FIG. 1).

| | PRIOR ART | COMPOSITION |
|---|---|---|
| | COMPOSI- | OF THE |
| INGREDIENT | TION | INVENTION |
| COCOS NUCIFERA | — | 0.95 |
| (COCONUT) OIL | | |
| MINERAL OIL | 1.5 | — |
| GUAR | — | 1 |
| HYDROXYPROPYLTRIMONIUM | | |
| CHLORIDE | | |
| AMODIMETHICONE (AND) | 0.5 | — |
| TRIDECETH-6 (AND) | | |
| CETRIMONIUM CHLORIDE | | |
| GLYCERIN | 0.5 | 0.5 |
| CETEARYL ALCOHOL | 4.2 | 4.2 |
| CETRIMONIUM CHLORIDE | 1.5 | 1.5 |
| BEHENTRIMONIUM CHLORIDE | 0.6 | 0.6 |
| PRESERVATIVE, FRGRANCE, | 0.27 | 0.27 |
| pH ADJUSTERS | | |
| WATER | QS | QS |

As demonstrated in the photographs of FIG. 1, the composition of the present invention provided better curl definition with more regularity in the curl shape until the ends of the hair as compared to the prior art composition without guar.

### EXAMPLE 5

A non-limiting example regarding the preparation of the composition of Examples 1 to 3, could be as follows:

(1) Adding 50% of the total amount of water in a recipient;

(2) Dissolving the preservatives;

(3) Adding the guar hydroxypropyltrimonium chloride and dispersing well, heating until 70° C.;

(4) Mixing well at 60° C., the cetearyl alcohol, coconut oil, behentrimonium chloride and cetrimonium chloride;

(5) Emulsifying for10 minutes;

(6) Starting the cooling step;

(7) Adding glycerin;

(8) Adding the remaining water

(9) Adding the fragrance at 40° C.;

(10) Adjusting the pH.

### EXAMPLE 6

A test was conducted in order to evaluate different modes of uses (routines using a serum formula with the inventive composition and traditional routine).

| | Performance of the | Performance of the |
|---|---|---|
| | traditional routine | traditional routine |
| | 1. Shampoo | 1. Shampoo |
| | 2. Conditioner | 2. Conditioner |
| | 3. Inventive Hair care | 3. Inventive Hair |
| | composition | care composition |
| | in visual aspect | in sensorial aspect |
| Routine 1: | Routine using serum | Routine using serum |
| 1. Shampoo* | mixed resulted in: | resulted in: |
| 2. Conditioner* | More curl definition | More smoothness |
| 3. Inventive Leave- | More curl regularity | More softness |
| on Hair care | More discipline | |
| composition + | More detangling | |
| Serum (mixed 2:1) | | |
| Routine 2: | Routine using serum | Routine using serum |
| 1. Shampoo | resulted in: | resulted in: |
| 2. Conditioner | More curl definition | More suppleness |
| 3. Inventive Leave- | More curl regularity | More lightness |
| on Hair care | More shine | |
| composition | Wet look | |
| 4. Serum | | |
| Routine 3 | Routine using a mask | Routine using serum |
| 1. Mask* | before shampoo and | resulted in: |
| 2. Shampoo | serum at the end | Less dry ends |
| 2. Conditioner | resulted in: | More softness |
| 3. Inventive Leave- | More curl definition | |
| on Hair care | More curl regularity | |
| composition | Less volume | |
| 4. Serum | Styling effect | |

*conventional shampoo, conditioner and mask

The serum formulation comprises the formula below:

| INGREDIENT | CONCENTRATION |
|---|---|
| CAPRYLIC/CAPRIC TRIGLYCERIDE | 0.4 |
| PLANT OILS | 0.3 |
| PLANT FLOWER EXTRACTS | 0.02 |
| DIMETHICONOL | 7.35 |
| VITAMINS, PRESERVATIVES, | <0.5 |
| FRAGRANCE | |
| CYCLOPENTASILOXANE | QS |

The results above show that when the inventive composition was used in a different routine, from the traditional routine (after a shampoo/conditioner routine), it was surprisingly found that the treated hair showed better curl regularity, better discipline, better curl definition, more suppleness, smoothness and shine, etc . . .

### EXAMPLE 7

A test was conducted in order to evaluate the performance of the hair care composition of the present invention in view of the examples below:

| | Example A | Example B | |
|---|---|---|---|
| | (composition of | (composition of | |
| | example 1 without | example 1 with guar | |
| | guar and with | and mineral oil - no | Example C |
| | coconut oil) | coconut oil) | (benchmark)* |
| Performance of | Use of the invention | Use of the invention | Use of the invention |
| the invention in | composition resulted in: | composition resulted in: | composition resulted in: |
| Wet Hair | More curl definition | Less frizziness | Less oiliness |
| | More curl regularity | Same preference | More curl definition |
| | Model preference | by panelists | More regularity |
| | | | More stickiness |
| Performance of | Use of the invention | Use of the invention | Use of the invention |
| the invention in | composition resulted in: | composition resulted in: | composition resulted in: |
| Dry Hair | Model preference | Less frizziness | More discipline |
| | More wet look | More discipline | Same curl |
| | | Same preference | definition |
| | | by panelists | Less frizziness |
| | | Same curl regularity | Model preference |

The Example C composition (commercial) comprises aqua, cetearyl alcohol, stearamidopropyl dimethylamine, dimethiconol, glycerin, parfum, lactic acid, paraffinum liquidum, methylparaben, petrolatum, dmdm hydantoin, disodium edta, cyclopentasiloxane, methoxypolyoxymethylene melamine, arginine, trimethylsiloxysilicate, benzyl benzoate, butylphenyl methylpropional, citronellol, geraniol, hexyl cinnamal, limonene, linalool.

The results above show that the curly hair treated with the inventive composition was less frizzy, more disciplined, and had curl regularity and curl definition that were on parity or even better compared to the hair treated with the prior art compositions.

1. A leave-on hair care composition comprising:
(a) about 0.9 to about 1.5% by weight of a cationic polymer;
(b) about 0.8 to about 5% by weight of a vegetal oil;
(c) about 3 to about 7% by weight of a fatty alcohol; and
(d) about 0.35 to about 5% by weight of at least two cationic surfactants;
all weights relative to the total weight of the composition.

2. The composition according to claim 1, wherein the cationic polymer is selected from quaternary ammonium guar, polymer derivatives of diallyldimethyl ammonium chloride (“poly-DADMAs”), methacrylamido-propyltrimethylammonium chloride (“poly-MAPTACs”), and mixtures thereof.

3. The composition according to claim 1, wherein the cationic polymer is guar hydroxypropyltrimonium chloride.

4. The composition according to claim 1, wherein the amount of cationic polymer is from about 0.9% to about 1.5% by weight, relative to the total weight of the composition.

5. The composition according to claim 1, wherein the vegetal oil is selected from acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, zedoary oil, and mixtures thereof.

6. The composition according to claim 1, wherein the vegetal oil is coconut oil.

7. The composition according to claim 1, wherein the amount of vegetal oil is from about 0.8% to about 5 by weight, relative to the total weight of the composition.

8. The composition according to claim 1, wherein the fatty alcohol is selected from linear, branched saturated/unsaturated fatty alcohols comprising from 6 to 60 carbon atoms.

9. The composition according to claim 1, wherein the fatty alcohol is cetearyl alcohol.

10. The composition according to claim 1, wherein the amount of fatty alcohol may range is from about 4.3% to about 7% by weight, relative to the total weight of the composition.

11. The composition according to claim 1, wherein the cationic surfactant is selected from quaternary ammonium salts.

12. (canceled)

13. The composition according to claim 1, wherein the amount of cationic surfactants is from about 0.35% to about 5% by weight, relative to the total weight of the composition.

14. The composition according to claim 1, wherein the ratio of cationic polymer to vegetal oil is from about 1:1 to about 1:1.6.

15. (canceled)

16. The composition according to claim 1, further comprising cosmetically acceptable ingredients selected from perfume/fragrance, polymers, preserving agents, solvents, actives, surfactants, fat materials, vitamins, vegetal extracts, emollients, vegetal oils, pH-adjusting, softeners, humectants, opacifiers, stabilizers, emollients, silicones, active agents and mixtures thereof.

17. The composition according to claim 1, in the form of an O/W (oil in water) emulsion.

18. The composition according to claim 1 having a viscosity from about 1.055 mPa·s to about 1.895 mPa·s.

19. (canceled)

20. (canceled)

21. A method of controlling fizz of curly hair comprising contacting the hair with the leave-on hair care composition according to claim 1.

22. A hair treatment agent comprising:
(1) a leave-on hair care composition comprising:
(a) about 0.9 to about 1.5% by weight of a cationic polymer;
(b) about 0.8 to about 5% by weight of a vegetal oil;
(c) about 3 to about 7% by weight of a fatty alcohol; and
(d) about 0.35 to about 5% by weight of at least two cationic surfactants;
all weights relative to the total weight of the leave-on hair care composition; and
(2) a serum composition comprising at least one silicone in an amount of from about 80 to about 100% by weight, relative to the total weight of the serum composition.

23. (canceled)

24. (canceled)

25. The hair-treatment agent according to claim 22, wherein the at least one silicone in the serum composition is selected from dimethicone, dimethiconol, cyclopentasiloxane, amino silicones, and mixtures thereof.

26. An article of manufacture for providing a hair care system for the care of curly hair, the article comprising:
(1) a leave-on hair care composition comprising:
(a) 0.9 to 1.5% by weight of a cationic polymer;
(b) 0.8 to 5% by weight of a vegetal oil;
(c) 3 to 7% by weight of a fatty alcohol; and
(d) 0.35 to 5% by weight of at least two cationic surfactants; and
(2) a serum composition containing at least one silicone;
wherein (1) and (2) are either mixable prior to application to hair or are can be applied separately and sequentially to hair in any order.

27. (canceled)
